(19) United States
(12) Patent Application Publication
(10) Pub. No.: US 2026/0358015 A1
(43) Pub. Date: Nov. 12, 2026

(54) VENTILATION SYSTEM

(71) Applicant: Panasonic Intellectual Property Management Co., Ltd., Osaka (JP)

(72) Inventors: Yuuji OZAKI, Kanagawa (JP); Kazuhiro TANIGUCHI, Chiba (JP); Ken YAMAMOTO, Osaka (JP); Tatsuya WATANABE, Kyoto (JP)

(21) Appl. No.: 19/125,168

(22) PCT Filed: Oct. 30, 2023
(86) PCT No.: PCT/JP2023/039153

§ 371 Date: Apr. 28, 2025

(30) Foreign Application Priority Data
Nov. 10, 2022 (JP) 2022-180579

(51) Int. Cl. F24F 11/72; F24F 13/06; F24F 13/08; F24F 110/10

(52) U.S. Cl. CPC F24F 11/72; F24F 13/06; F24F 13/08; F24F 2110/10

(57) ABSTRACT

A ventilation system includes a nozzle unit and a ventilation device. The nozzle unit includes at least two nozzles. The at least two nozzles each include a housing having a hollow elongated shape extending in a first direction, the at least two nozzles being arranged side by side in alignment with a second direction. The housing has a first surface part and a second surface part facing each other in a third direction. The first surface part has a ventilation port extending in alignment with the first direction. The ventilation port is configured to allow air sent into the housing to be blown to the outside therethrough. The ventilation device is located at a side of the second surface part of the housing with respect to the nozzle unit and is configured to blow second conditioned air from a first end toward a second end of the nozzle unit.

### TECHNICAL FIELD

The present disclosure relates to a ventilation system.

### BACKGROUND ART

Patent Literature 1 describes a ventilation device including a plurality of nozzles having the same length. Each nozzle has an inflow port and a vent. High-pressure air flows into the nozzles through the inflow ports, and the high-pressure air in the nozzles blows out through the vents. The plurality of nozzles are disposed with a gap therebetween such that the respective vents of the nozzles are on the same plane. The gap forms, outside the nozzles, a path for air drawn in streams of the high-pressure air blowing out through the vents. The ventilation device includes a damper mechanism capable of changing an opening area of the inflow port of each nozzle. The ventilation device adjusts the opening area of the inflow port of each nozzle, thereby adjusting a ventilation range.

In such a ventilation device of Patent Literature 1, the nozzles each have to be provided with the damper mechanism capable of changing the opening area of the inflow port thereof to allow a ventilation region to be changed. This results in a complex nozzle structure.

### CITATION LIST

### Patent Literature

Patent Literature 1:JP 2018-003658 A

### SUMMARY OF INVENTION

It is an object of the present disclosure to provide a ventilation system capable of changing a ventilation region with a simplified nozzle structure.

A ventilation system of an aspect of the present disclosure is installed in a space in which an air conditioning device blows first conditioned air. The ventilation system includes a nozzle unit and a ventilation device. The nozzle unit includes at least two nozzles each including a housing having a hollow elongated shape extending in a first direction, the at least two nozzles being arranged side by side in alignment with a second direction intersecting the first direction. The ventilation device is configured to suck in the first conditioned air and blow out second conditioned air. The housing has a first surface part and a second surface part facing each other in a third direction intersecting the first direction and the second direction. The first surface part has a ventilation port extending in the first direction. The ventilation port is configured to allow air sent into the housing to be blown to an outside of the housing through the ventilation port. The ventilation device is located at a side of the second surface part of the housing with respect to the nozzle unit and configured to blow the second conditioned air from a first end toward a second end in the first direction of the nozzle unit.

### BRIEF DESCRIPTION OF DRAWINGS

FIG. 1 is a perspective view of a ventilation system of an embodiment;

FIG. 2 is a front view of a main part of the ventilation system;

FIG. 3 is a perspective view of the main part of the ventilation system;

FIG. 4 is a front view of a ventilation unit included in the ventilation system;

FIG. 5 is a sectional view of a part of the ventilation system;

FIG. 6 is a plan view of an upper end of a nozzle included in the ventilation system;

FIG. 7 is a view of a part of the ventilation unit included in the ventilation system;

FIG. 8 is a block diagram of a part of the ventilation system;

FIG. 9 is an illustrative view of operation of the ventilation system during heating operation;

FIG. 10 is an illustrative view of warm airflow in the ventilation system ;

FIG. 11 is an illustrative view of operation of the ventilation system during cooling operation;

FIG. 12 is an illustrative view of cool airflow in the ventilation system;

FIG. 13 is a sectional view of a part of a ventilation system of a second variation;

FIG. 14 is a sectional view of a part of a ventilation system of a third variation;

FIG. 15 is a block diagram of a part of the ventilation system of the third variation;

FIG. 16 is a block diagram of a part of a ventilation system of a fourth variation; and

FIG. 17 is a sectional view of a part of a ventilation system of a fifth variation.

### DESCRIPTION OF EMBODIMENTS

The present embodiment generally relates to ventilation systems. More specifically, the present disclosure relates to a ventilation system including at least two nozzles each having a hollow elongated shape, the at least two nozzles being arranged parallel to each other.

Note that the embodiment described below is a mere example of embodiments of the present disclosure. The present disclosure is not limited to the embodiment described below, and various modifications may be made to the embodiment described below as long as the effect of the present disclosure is produced.

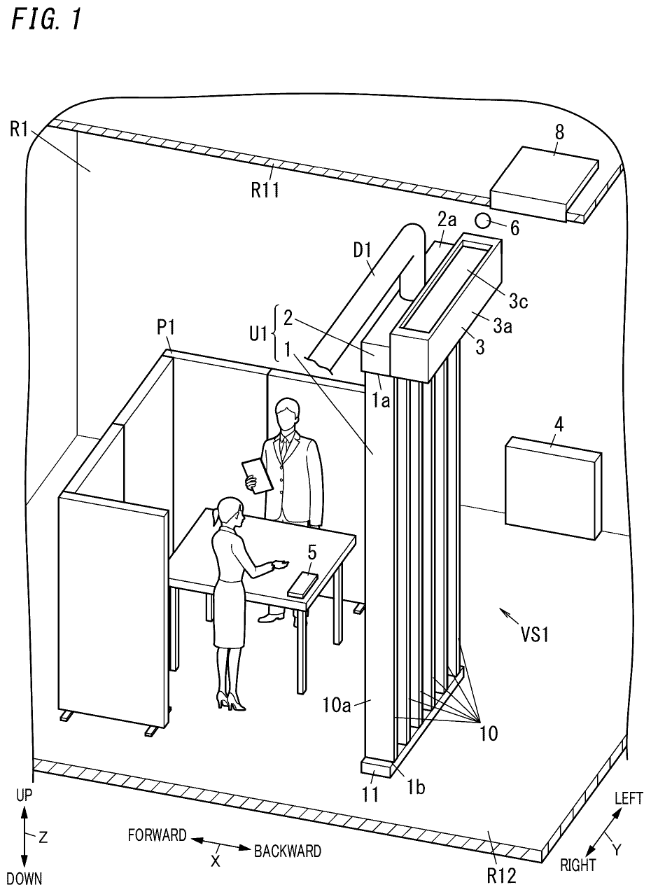

Moreover, in the following description, unless otherwise indicated, X, Y, and Z axes orthogonal to one another are defined as shown in FIG. 1. For convenience, one of both directions along the X axis is defined as a forward direction, and the other is defined as a backward direction. Further, one of both directions along the Y axis is defined as a left direction, and the other is defined as a right direction. Furthermore, one of both directions along the Z axis is defined as an upward direction, and the other is defined as a downward direction.

### EMBODIMENT

### (1) Overview

FIG. 1 shows a ventilation system VS1 of the present embodiment. The ventilation system VS1 is used in a facility such as an office building, an office, a retail establishment, a factory, or a commercial facility. Moreover, the ventilation system VS1 may be used in, for example, a dwelling unit of a multi-family dwelling house or a detached house. The ventilation system VS1 is assumed to be installed in a building such as a facility or a dwelling house but may be installed in a structural object other than the building.

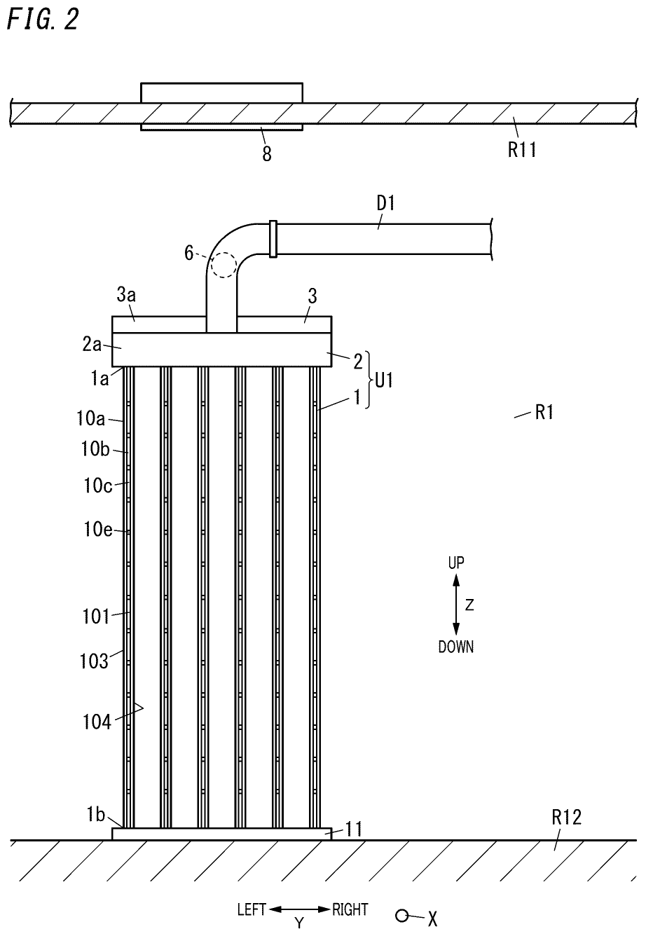

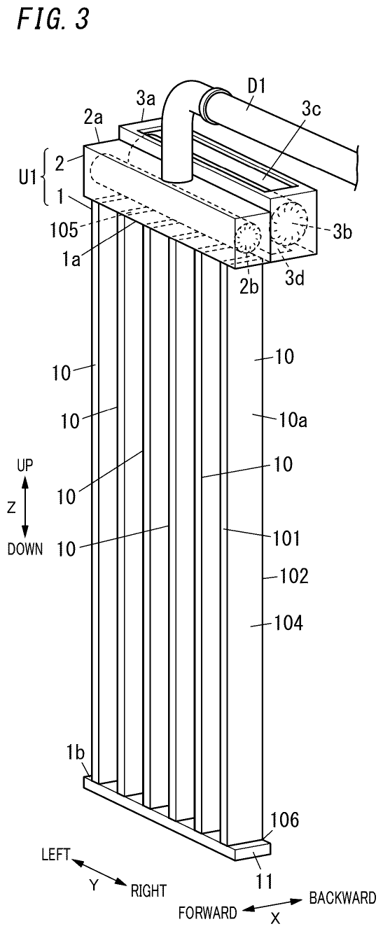

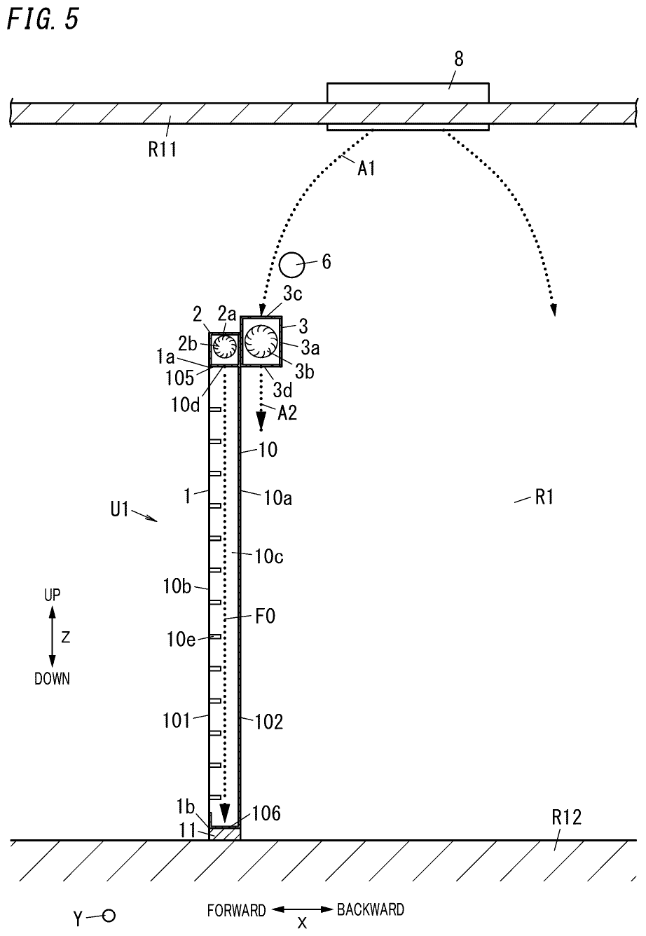

The ventilation system VS1 of the present embodiment is installed in a space R1 in which an air conditioning device 8 blows first conditioned air A1 (see FIG. 5). The ventilation system VS1 includes a nozzle unit 1 and a ventilation device 3. The nozzle unit 1 includes at least two nozzles 10. The at least two nozzles 10 each include a housing 10a having a hollow elongated shape extending in alignment with a first direction. The at least two nozzles 10 are arranged side by side in alignment with a second direction intersecting the first direction. The ventilation device 3 sucks in the first conditioned air A1 and blows out second conditioned air A2 (see FIG. 5). The housing 10a has a first surface part 101 (see FIG. 3) and a second surface part 102 (see FIG. 3) facing each other in a third direction intersecting the first direction and the second direction. The first surface part 101 has a ventilation port 10b (see FIG. 2) extending in alignment with the first direction. The ventilation port 10b allows air sent into the housing 10a to be blown to the outside of the housing 10a therethrough. The ventilation device 3 is located at the side of the second surface part 102 of the housing 10a with respect to the nozzle unit 1 and blows the second conditioned air A2 from a first end toward a second end in the first direction of the nozzle unit 1.

In the ventilation system VS1 having the configuration described above, the nozzle unit 1 draws in the second conditioned air A2 blown out of the ventilation device 3 in the first direction, thereby allowing a ventilation region of the second conditioned air A2 blown out of the first surface part 101 to be changed. That is, the ventilation system VS1 enables the ventilation region to be changed with a simplified structure of each nozzle 10.

Note that in the present embodiment, the first direction corresponds to a vertical direction (up/down direction) along the Z-axis, the second direction corresponds to the left/right direction along the Y-axis, and the third direction corresponds to the forward/backward direction along the X-axis.

Moreover, in the present embodiment, the first end of the nozzle unit 1 corresponds to an upper end 1a of the nozzle unit 1, and the second end of the nozzle unit 1 corresponds to a lower end 1b of the nozzle unit 1.

### (2) Details

As shown in FIG. 1, the ventilation system VS1 is installed in the space R1. The space R1 is a space, such as a working space, a meeting room, a rest room, a waiting room, a reception room, or a living room in which a person or people are present, and the air conditioning device 8 is installed in the space R1. The space R1 has an upper surface which is a ceiling R11, and the space R1 has a lower surface which is a floor R12.

The ventilation system VS1 includes the nozzle unit 1, the ventilation device 3, and the control device 4. The nozzle unit 1 includes six nozzles 10. Moreover, the ventilation system VS1 preferably further includes a nozzle ventilator 2. In this embodiment, the nozzle unit 1 and the nozzle ventilator 2 are included in a ventilation unit U1. Moreover, the ventilation system VS1 preferably further includes an operation device 5 and a temperature sensor 6.

Moreover, the six nozzles 10 included in the nozzle unit 1 each includes the housing 10a having a hollow elongated shape extending in alignment with the vertical direction. The six housings 10a are arranged side by side and spaced apart from each other in alignment with the left/right direction. As a result, the nozzle unit 1 can be deemed to have a plate shape extending along a Z-Y plane as a whole and can be used as a partition spatially partitioning the space R1. In FIG. 1, the nozzle unit 1 is installed in the space R1 together with a partition P1, thereby forming a space surrounded by the nozzle unit 1 and the partition P1 in the space R1.

### (2.1) Air Conditioning Device

The air conditioning device 8 is an air conditioner having a heating function and a cooling function and is installed by being embedded in the ceiling R11 as shown in FIG. 1. The air conditioning device 8 blows the first conditioned air Al into the space R1. In the present embodiment, the air conditioning device 8 has a ventilation port having a shape conforming to an outer peripheral edge of a housing of the air conditioning device 8 and blows the first conditioned air Al obliquely downward through the ventilation port. The air conditioning device 8 blows warm air as the first conditioned air A1 during heating operation. The air conditioning device 8 blows cool air as the first conditioned air A1 during cooling operation.

### (2.2) Ventilation Unit

The ventilation unit U1 is fixed to a lower surface of the ceiling R11, a T-bar, or the like with, for example, a hanger bolt or a wire (not shown). As shown in FIGS. 2 to 5, the ventilation unit U1 includes the nozzle unit 1 and the nozzle ventilator 2.

The nozzle unit 1 includes the six nozzles 10 aligned spaced apart from each other in the left/right direction and a base 11.

The nozzles 10 each have the housing 10a whose long sides extend in the vertical direction along the Z-axis and which has a hollow rectangular plate shape. The housing 10a has a front surface part 101, a rear surface part 102, a left surface part 103, a right surface part 104, an upper end surface part 105, and a lower end face part 106 which are each rectangular.

The front surface part 101 of the housing 10a has a rectangular opening formed as the ventilation port 10b, the rectangular opening having long sides extending in the vertical direction. The ventilation port 10b is formed at the center in the left/right direction in the front surface part 101 of the housing 10a. The respective housings 10a of the six nozzles 10 are aligned side by side in the left/right direction along the Y-axis and are spaced apart from each other. The left surface part 103 of the housing 10a of one nozzle 10 faces the right surface part 104 of the housing 10a of another nozzle 10 adjacent to the left of the one nozzle 10 while a gap provided between the left surface part 103 and the right surface part 104. The right surface part 104 of the housing 10a of one nozzle 10 faces the left surface part 103 of the housing 10a of another nozzle 10 adjacent to the right of the one nozzle 10 while a gap provided between the right surface part 104 and the left surface part 103. The housings 10a are made of, for example, a resin material but may be made of a light-weight metal material such as aluminum.

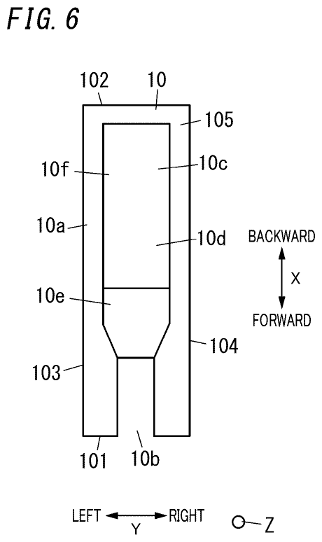

Each housing 10a has an internal space 10c formed in the interior thereof. The internal space 10c is surrounded by the front surface part 101, the rear surface part 102, the left surface part 103, the right surface part 104, the upper end surface part 105, and the lower end face part 106. As shown in FIG. 6, the upper end surface part 105 of the housing 10a has a suction port 10d. The internal space 10c communicates with the outside of the housing 10a via the suction port 10d. The lower end face part 106 of the housing 10a has an inner peripheral surface 10f facing the internal space 10c.

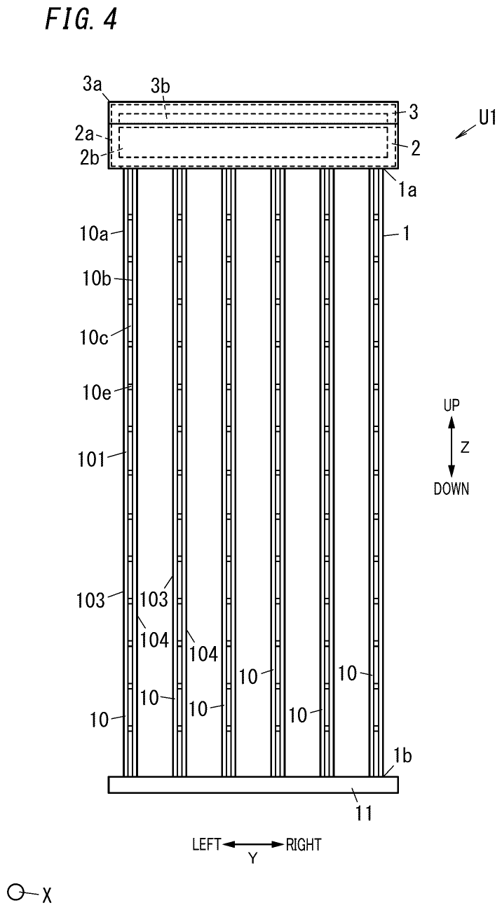

As shown in FIGS. 4, 5, and 6, the front surface part 101 of the internal space 10c has a rear surface provided with a plurality of fins 10e aligned side by side in the vertical direction at regular intervals. Each fin 10e has a plate shape extending rearward from the front surface part 101 of the internal space 10c and closes a front part of the internal space 10c (part of a front side portion of the internal space 10c) when viewed in a direction along the Z-axis. As shown in FIG. 4, when the housing 10a of each of the nozzles 10 is viewed from the front, the plurality of fins 10e are located to separate the ventilation port 10b at regular intervals along the Z-axis. The fins 10e have a function of rectifying air which is to be blown to the outside of the housing 10a through the ventilation port 10b.

The nozzle ventilator 2 is disposed at the upper end la of the nozzle unit 1. The nozzle ventilator 2 includes: a housing 2a in the shape of a hollow parallelepiped, and a fan 2b in the housing 2a. The fan 2b is preferably a cross-flow fan. The housing 2a is connected to a duct D1 and is supplied with air from the duct D1. Air blown out of the fan 2b by rotation of the fan 2b blows downward out of a lower surface of the housing 2a. The lower surface of the housing 2a faces the upper end surface parts 105 of the nozzles 10, and the air blown out of the fan 2b by the rotation of the fan 2b flows through the suction ports 10d of the nozzles 10 into the internal spaces 10c. That is, the nozzle ventilator 2 sends air through the suction port 10d of each nozzle 10 into the internal space 10c, thereby generating, in the internal space 10c, interior airflow FO (see FIG. 5) flowing downward from the suction port 10d. The interior airflow FO is rectified by the fins 10e in the internal space 10c and blows out forward through the ventilation port 10b of the front surface part 101 of the housing 10a. Note that as shown in FIG. 6, the width of the front portion of the internal space 10c in the left/right direction preferably narrows forward.

The base 11 has an elongated rectangular plate shape. The lower end 1b of the nozzle unit 1 is fixed to the base 11. That is, the lower end face parts 106 of the six housings 10a are fixed to the base 11 (see FIG. 5). The base 11 is mounted on the floor R12, and thereby, the six nozzles 10 are mounted on the floor R12 such that the housings 10a extend in the vertical direction.

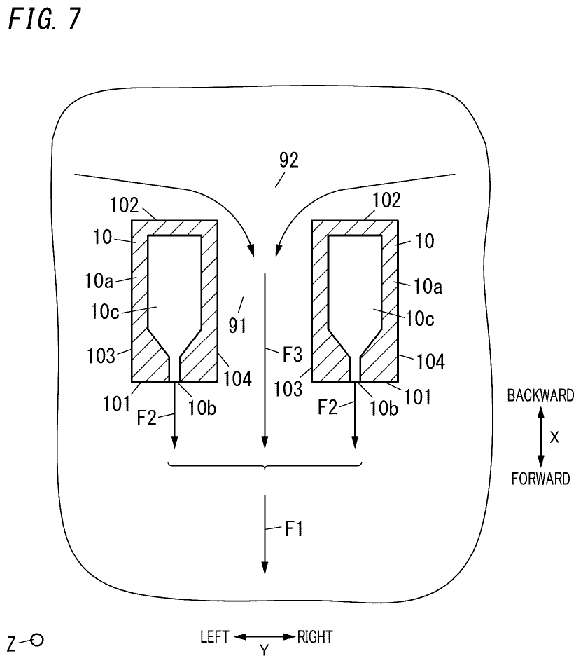

As shown in FIG. 7, the six nozzles 10 included in the nozzle unit 1 each blow air forward through the elongated ventilation port 10b of the front surface part 101 of the housing 10a, thereby generating nozzle airflow F2. Note that FIG. 7 shows arbitrary two nozzles 10 of the six nozzles 10 included in the nozzle unit 1. The arbitrary two nozzles 10 are disposed adjacent to each other in the left/right direction along the Y-axis.

In this embodiment, a draw-in pathway 91 shown in FIG. 7 is formed between the two nozzles 10 adjacent to each other in the left/right direction. The draw-in pathway 91 is a space provided between the right surface part 104 of the housing 10a of a left one of the two nozzles 10 in the left/right direction and a left surface part 103 of the housing 10a of a right one of the two nozzles 10 in the left/right direction such that the space is open forward and backward. When each of the two nozzles 10 arranged side by side produces the nozzle airflow F2 blowing out forward through the ventilation port 10b, a negative pressure is produced in the draw-in pathway 91, and air in a rear space 92 which is a space backward of the two nozzles 10 is drawn in the draw-in pathway 91 from the rear to the front. The air drawn in the draw-in pathway 91 from the rear to the front blows out forward through the draw-in pathway 91. The air blowing out forward through the draw-in pathway 91 produces draw-in airflow F3 flowing forward from the draw-in pathway 91.

As a result, at a front lower location of the nozzle unit 1, the draw-in airflow F3 is generated between the two streams of the nozzle airflow F2 produced by the two nozzles 10 adjacent to each other, and combination airflow F1 which is a combination of the streams of the nozzle airflow F2 and the draw-in airflow F3 is produced. The combination airflow F1 blows forward out of the nozzle unit 1.

The operation and deactivation of the fan 2b is controlled by a control device 4. Moreover, the control device 4 may control the rotational velocity of the fan 2b. In this case, when the flow rate of the combination airflow F1 is defined as the ventilation amount of the nozzle unit 1, the ventilation amount of the nozzle unit 1 increases as the rotational velocity of the fan 2b increases, and the ventilation amount of the nozzle unit 1 decreases as the rotational velocity of the fan 2b decreases.

### (2.3) Ventilation Device

The ventilation device 3 is a cross-flow fan, is fixed to the lower surface of the ceiling R11, a T-bar, or the like with, for example, a hanger bolt or a wire (not shown), and is disposed backward of the upper end la of the nozzle unit 1 (or backward of the nozzle unit 1). The ventilation device 3 sucks in the first conditioned air A1 blown out of the air conditioning device 8 and blows the second conditioned air A2 from the upper end (first end) 1a toward the lower end (second end) 1b of the nozzle unit 1 (see FIG. 5).

Specifically, as shown in FIG. 5, the ventilation device 3 includes: a housing 3a in the shape of a hollow parallelepiped; and a fan 3b in the housing 3a. The housing 3a has an upper surface having a suction port 3c, and the housing 3a has a lower surface having a ventilation port 3d. When the fan 3b rotates, the first conditioned air A1 is sucked through the suction port 3c into the housing 3a, and the second conditioned air A2 is blown out downward through the ventilation port 3d. The second conditioned air A2 blowing out downward through the ventilation port 3d flows downward from above at the back of the nozzle unit 1. In the present embodiment, the ventilation device 3 is preferably located in a flow path of the first conditioned air A1 blown out of the air conditioning device 8. In FIG. 5, the ventilation device 3 is located diagonally downward of the air conditioning device 8.

When the flow rate of the second conditioned air A2 at the ventilation port 3d is defined as the ventilation amount of the ventilation device 3, the ventilation amount of the ventilation device 3 increases as the rotational velocity of the fan 3b increases, and the ventilation amount of the ventilation device 3 decreases as the rotational velocity of the fan 3b decreases. The operation, deactivation, and rotational velocity of the fan 3b are controlled by the control device 4.

### (2.4) Operation Device

The operation device 5 includes a switch or a touch panel for an instruction on the operation of the ventilation device 3 and receives an operation given by a user. The operation device 5 transmits, to the control device 4, a ventilation operation signal according to the operation given by the user. Specifically, the operation device 5 receives operations regarding operation, deactivation, and the like of the ventilation system VS1. The control device 4 controls, based on the ventilation operation signal received from the operation device 5, the operation, the deactivation, and the ventilation amount during the operation of the ventilation device 3, and the operation and the deactivation of the nozzle ventilator 2.

The operation device 5 is, for example, a smartphone, a tablet computer, or a special-purpose terminal.

### (2.5) Temperature Sensor

As shown in FIG. 5, the temperature sensor 6 is installed in the vicinity of the air conditioning device 8 to detect the temperature of the first conditioned air A1 blown out of the air conditioning device 8. The temperature sensor 6 generates a temperature signal including a detection result of the temperature of the first conditioned air A1 and outputs the temperature signal to the control device 4. The control device 4 controls, based on the temperature signal received from the temperature sensor 6, a ventilation amount during operation of the ventilation device 3.

In the present embodiment, the temperature sensor 6 is preferably located in the flow path of the first conditioned air Al blown out of the air conditioning device 8. In FIG. 5, the ventilation device 3 is located obliquely downward of the air conditioning device 8 and between the air conditioning device 8 and the ventilation device 3 in the vertical direction.

### (2.6) Control Device

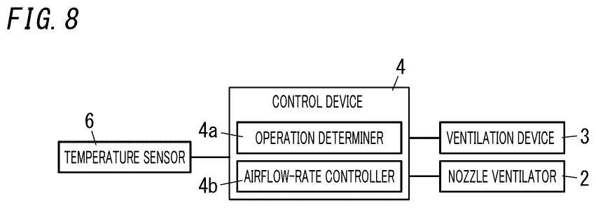

FIG. 8 is a block diagram relating to control performed by the control device 4. The control device 4 performs wired or wireless communication with the ventilation device 3, thereby controlling the operation, deactivation, and ventilation amount of the ventilation device 3. Moreover, the control device 4 performs wired or wireless communication with the nozzle ventilator 2, thereby controlling the operation and deactivation of the nozzle ventilator 2. Moreover, the control device 4 performs wired or wireless communication with the operation device 5 and the temperature sensor 6, thereby acquiring the ventilation operation signal from the operation device 5 and the temperature signal from the temperature sensor 6. Note that the wired communication is wired communication, for example, over a twisted pair cable, a dedicated communication line, or a local area network (LAN) cable. The wireless communication is wireless communication compliant with the standard of, for example, Wi-Fi (registered trademark), Bluetooth (registered trademark), ZigBee (registered trademark), or low power radio (specified low power radio) requiring no license.

Thus, the control device 4 can switch, based on the operation given by the user and received by the operation device 5 and the detection result by the temperature sensor 6, between the operation and the deactivation of the ventilation device 3 and adjust the ventilation amount during the operation of the ventilation device 3. Moreover, the control device 4 can switch, based on the operation given by the user and received by the operation device 5, between the operation and the deactivation of the nozzle ventilator 2.

The control device 4 preferably includes a computer system. That is, in the control device 4, a processor of a central processing unit (CPU), a micro processing unit (MPU), or the like reads and executes a program stored in memory, thereby implementing some or all functions of the control device 4. The control device 4 includes, as a principal hardware component, a processor that operates in accordance with a program. There is no restriction on the type of the processor as long as the processor can implement a function by executing a program. The processor includes one or more electronic circuits including a semiconductor integrated circuit (IC), or a large scale integration (LSI) circuit. The integrated circuit such as IC or LSI mentioned herein may be referred to in another way, depending on the degree of the integration and includes integrated circuits called system LSI, very-large-scale integration (VLSI), or ultra-large-scale integration (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. The plurality of electronic circuits may be collected on one chip or may be distributed on a plurality of chips. The plurality of chips may be collected in one device or may be distributed in a plurality of devices.

Moreover, the control device 4 may be embodied by a single computer device or may be implemented by a plurality of computer devices linked to each other. Moreover, the control device 4 may be configured as a cloud computing system.

The control device 4 of the present embodiment controls the ventilation amount of the ventilation device 3 on the basis of the detection result by the temperature sensor 6, thereby performing ventilation control according to the operation state of the air conditioning device 8. The ventilation control in the ventilation system VS1 will be described in detail below.

### (2.7) Ventilation Control of Ventilation Device

In the ventilation system VS1, the control device 4 controls the ventilation amount of the second conditioned air A2 by the ventilation device 3, thereby adjusting a region to which the airflow generated in front of the nozzle unit 1 flows. In the present embodiment, the control device 4 controls such that the nozzle ventilator 2 is in the operating state, and then, the control device 4 adjusts the ventilation amount of the ventilation device 3, thereby allowing the region (ventilation region) to which the airflow generated in front of the nozzle unit 1 flows to be changed.

Specifically, the control device 4 includes an operation determiner 4a, and an airflow-rate controller 4b as shown in FIG. 8.

The operation determiner 4a determines the operation state of the air conditioning device 8. In the present embodiment, the operation determiner 4a monitors, based on the temperature signal received from the temperature sensor 6, the temperature of the first conditioned air A1. The operation determiner 4a determines, based on the temperature of the first conditioned air A1, the operation state of the air conditioning device 8. In particular, the operation determiner 4a preferably determines whether the operation state of the air conditioning device 8 is the heating operation or the cooling operation. If the temperature of the first conditioned air A1 is higher than or equal to a threshold, the operation determiner 4a determines that the operation state of the air conditioning device 8 is the “heating operation”. If the temperature of the first conditioned air A1 is lower than the threshold, the operation determiner 4a determines that the operation state of the air conditioning device 8 is the “cooling operation”. Alternatively, the operation determiner 4a may further acquire at least one piece of data on a room temperature or an outdoor temperature, and based on the temperature of the first conditioned air A1 and the room temperature, on the temperature of the first conditioned air A1 and the outdoor temperature, or on the temperature of the first conditioned air A1, the room temperature, and the outdoor temperature, the operation determiner 4a may determine the operation state of the air conditioning device 8.

The airflow-rate controller 4b controls, based on a determination result of the operation state of the air conditioning device 8, the ventilation amount of the second conditioned air A2 by the ventilation device 3. In particular, the airflow-rate controller 4b preferably controls such that the ventilation amount in the case of the operation state being the heating operation is larger than the ventilation amount in the case of the operation state being the cooling operation. The ventilation control during the heating operation and the ventilation control during the cooling operation will be described in detail below.

### (2.7.1) Ventilation Control during Heating Operation

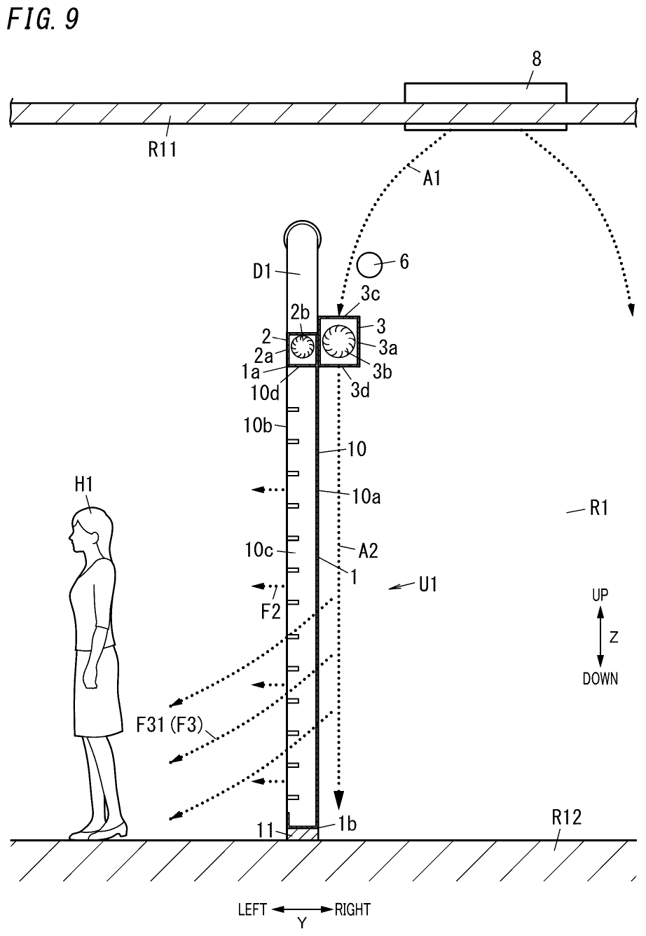

In FIG. 9, the air conditioning device 8 performs the heating operation, thereby blowing warm air as the first conditioned air A1 downward. Moreover, the nozzle ventilator 2 is in the operation state, and the ventilation device 3 is also in the operation state.

In this case, the operation determiner 4a of the control device 4 determines, based on the temperature signal received from the temperature sensor 6, that the operation state of the air conditioning device 8 is the “heating operation”. If the operation state of the air conditioning device 8 is the “heating operation”, the airflow-rate controller 4b of the control device 4 controls such that the ventilation amount of the ventilation device 3 is “large”. The ventilation amount “large” of the ventilation device 3 is larger than the ventilation amount “small” of the ventilation device 3 during the cooling operation which will be described later. When the ventilation amount of the ventilation device 3 is “large”, the flow rate of the second conditioned air A2 blown downward out of the ventilation device 3 which has sucked in the first conditioned air A1 is high. At this time, the second conditioned air A2 swiftly flows, at the back of the nozzle unit 1, from the upper end la toward the lower end 1b of the nozzle unit 1, and most of the second conditioned air A2 reaches the lower end 1b of the nozzle unit 1.

Moreover, the nozzle ventilator 2 is in the operation state, and the nozzle airflow F2 blows forward out of a front surface of the nozzle unit 1 (through the ventilation port 10b of each nozzle 10). Thus, part of the second conditioned air A2 flowing downward from above at the back of the nozzle unit 1 is drawn in the nozzle airflow F2 blowing out of the front surface of the nozzle unit 1 and is thus drawn toward the front. The second conditioned air A2 drawn toward the front forms drawn-in airflow F31 as the draw-in airflow F3 passing through two nozzles 10 adjacent to each other in the left/right direction. A speed vector of the draw-in airflow F31 is a resultant vector of a speed vector of the second conditioned air A2 and a speed vector of the nozzle airflow F2, and the draw-in airflow F31 travels in an obliquely forward and downward direction.

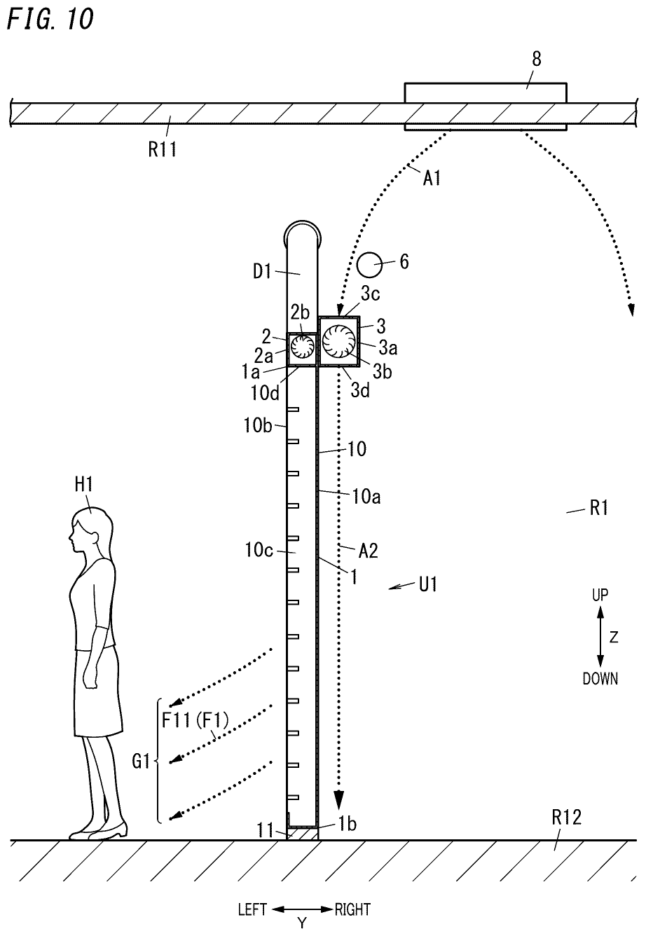

As the combination airflow F1 of the draw-in airflow F31 and the nozzle airflow F2, warm airflow F11 shown in FIG. 10 is generated, and the warm airflow F11 travels in a forward and obliquely downward direction. In this embodiment, when the air conditioning device 8 performs the heating operation, the ventilation amount of the ventilation device 3 is “large” and the flow rate of the second conditioned air A2 blown downward out of the ventilation device 3 is large. As a result, the draw-in airflow F31 is mainly generated in a region closer to the lower end 1b than to the upper end 1a of the nozzle unit 1 (i.e., at a lower portion of the nozzle unit 1) (see FIG. 9). Thus, the warm airflow F11 blows forward mainly out of the lower portion of the nozzle unit 1, and a ventilation region G1 of the warm airflow F11 is at a lower location in front of the nozzle unit 1.

That is, the warm airflow F11 travels toward the vicinity of the feet of a person H1 in the space R1, and thus, the ventilation system VS1 can improve the comfort of the person H1 by heating. Moreover, the warm airflow F11 blows out toward the lower portion of the space R1, and thus, the ventilation system VS1 can generate a convection flow in the space R1 during heating, thereby making the temperature distribution in the space R1 uniform.

### (2.7.2) Ventilation Control during Cooling Operation

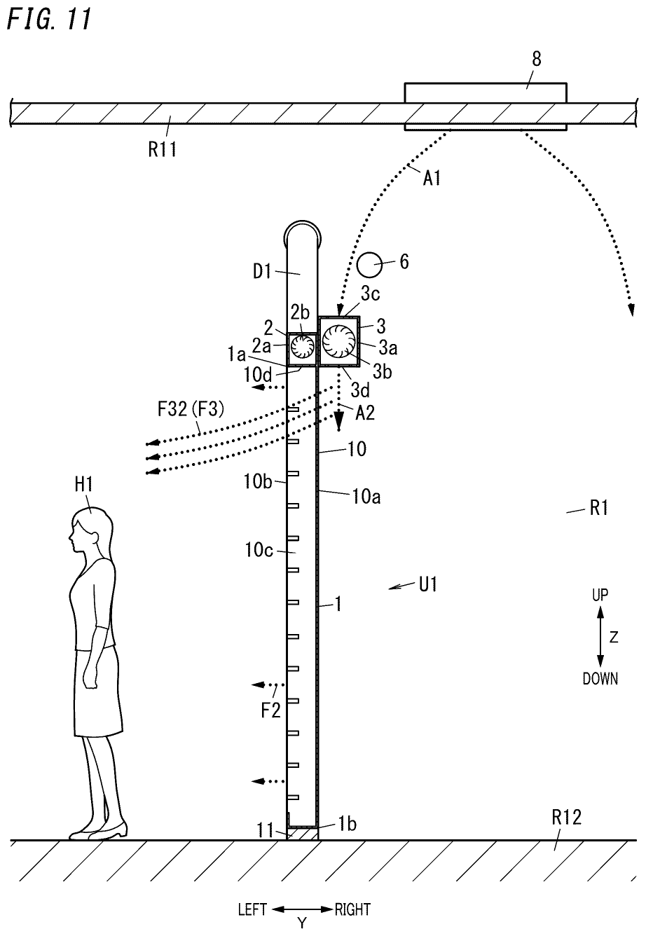

In FIG. 11, the air conditioning device 8 performs the cooling operation, thereby blowing cool air as the first conditioned air A1 downward. Moreover, the nozzle ventilator 2 is in the operation state, and the ventilation device 3 is also in the operation state.

In this case, the operation determiner 4a of the control device 4 determines, based on the temperature signal received from the temperature sensor 6, that the operation state of the air conditioning device 8 is the “cooling operation”. If the operation state of the air conditioning device 8 is the “cooling operation”, the airflow-rate controller 4b of the control device 4 controls such that the ventilation amount of the ventilation device 3 is “small”. The ventilation amount “small” of the ventilation device 3 is smaller than the ventilation amount “large” of the ventilation device 3 during the heating operation. When the ventilation amount of the ventilation device 3 is “small”, the flow rate of the second conditioned air A2 blown downward out of the ventilation device 3 which has sucked in the first conditioned air A1 is small. At this time, the second conditioned air A2 flows, at the back of the nozzle unit 1, from the upper end 1a to the lower end 1b of the nozzle unit 1, but most of the second conditioned air A2 does not reach the lower end 1b of the nozzle unit 1.

Moreover, the nozzle ventilator 2 is in the operation state, and the nozzle airflow F2 blows forward out of a front surface of the nozzle unit 1 (through the ventilation port 10b of each nozzle 10). Thus, part of the second conditioned air A2 flowing downward from above at the back of the nozzle unit 1 is drawn in the nozzle airflow F2 blowing out of the front surface of the nozzle unit 1 and is thus drawn toward the front. The second conditioned air A2 drawn toward the front forms drawn-in airflow F32 as the draw-in airflow F3 passing through two nozzles 10 adjacent to each other in the left/right direction. A speed vector of the draw-in airflow F32 is a resultant vector of a speed vector of the second conditioned air A2 and a speed vector of the nozzle airflow F2, and the draw-in airflow F32 travels in an obliquely forward and downward direction.

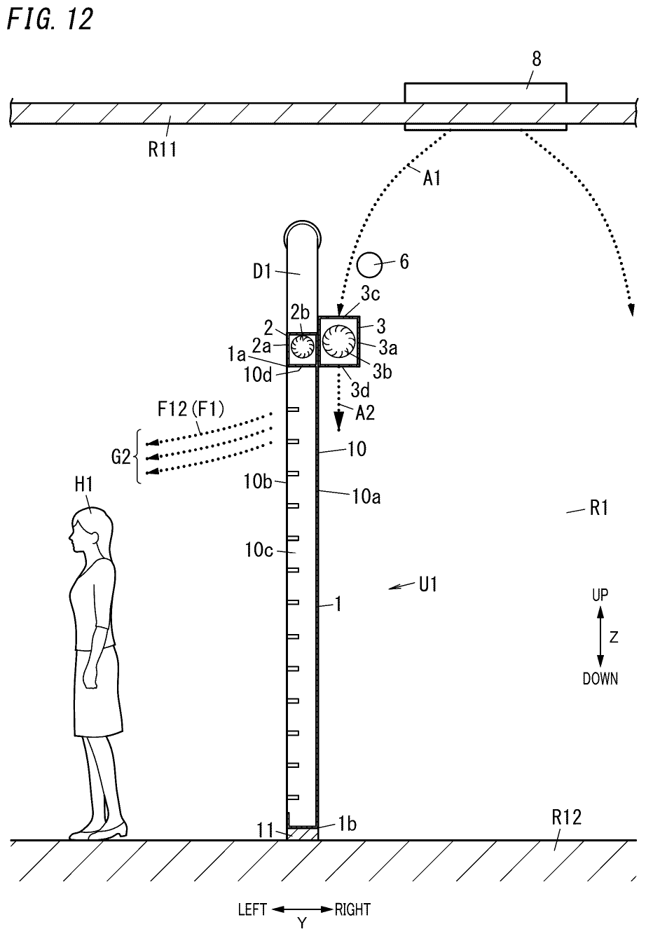

As the combination airflow F1 of the draw-in airflow F32 and the nozzle airflow F2, cool airflow F12 shown in FIG. 12 is generated, and the cool airflow F12 travels in forward and obliquely downward direction. In this embodiment, when the air conditioning device 8 performs the cooling operation, the ventilation amount of the ventilation device 3 is “small” and the flow rate of the second conditioned air A2 blown downward out of the ventilation device 3 is small. As a result, the draw-in airflow F32 is mainly generated in a region closer to the upper end la than to the lower end 1b of the nozzle unit 1 (i.e., at an upper portion of the nozzle unit 1) (see FIG. 11). Thus, the cool airflow F12 blows forward mainly out of the upper portion of the nozzle unit 1, and a ventilation region G2 of the cool airflow F12 is at an upper location in front of the nozzle unit 1.

That is, the cool airflow F12 travels toward the vicinity of an area above the head of the person H1 in the space R1, and thus, the cool airflow F12 does not directly reach the person H1, and the ventilation system VS1 can improve the comfort of the person H1 by cooling. Moreover, the cool airflow F12 blows out toward the upper portion of the space R1, and thus, the ventilation system VS1 can generate a convection flow in the space R1 during cooling, thereby making the temperature distribution in the space R1 uniform.

### (2.7.3) Advantages

The ventilation system VS1 described above enables the ventilation region to be changed with a simplified structure of each nozzle 10.

Moreover, the ventilation system VS1 includes the operation determiner 4a and the airflow-rate controller 4b and can thus send airflow to a ventilation region according to the operation state of the air conditioning device 8.

Moreover, the operation determiner 4a determines whether the operation state of the air conditioning device 8 is the heating operation or the cooling operation. Furthermore, the airflow-rate controller 4b controls such that the ventilation amount of the ventilation device 3 in the case of the operation state being the heating operation is larger than the ventilation amount of the ventilation device 3 in the case of the operation state being the cooling operation. As a result, the ventilation system VS1 can send the warm airflow F11 to the ventilation region G1 during the heating operation of the air conditioning device 8 and can send the cool airflow F12 to the ventilation region G2 during the cooling operation of the air conditioning device 8.

Moreover, the ventilation system VS1 further includes the temperature sensor 6 for detecting the temperature of the first conditioned air A1, and the operation determiner 4a determines the operation state on the basis of the temperature of the first conditioned air A1. As a result, the operation determiner 4a can accurately determine the operation state of the air conditioning device 8.

Moreover, the six nozzles 10 included in the nozzle unit 1 each includes the housing 10a having a hollow elongated shape extending in alignment with the vertical direction. The ventilation device 3 blows the second conditioned air A2 from the upper end 1a toward the lower end 1b of the nozzle unit 1. Furthermore, the airflow-rate controller 4b controls such that the ventilation amount of the ventilation device 3 in the case of the operation state being the heating operation is larger than the ventilation amount of the ventilation device 3 in the case of the operation state being the cooling operation. As a result, the ventilation system VS1 can generate the warm airflow F11 which travels toward the vicinity of the feet of the person H1 in the space R1 while the air conditioning device 8 performs the heating operation. Moreover, the ventilation system VS1 can generate the cool airflow F12 which travels toward the vicinity of an area above the head of the person H1 in the space R1 while the air conditioning device 8 performs the cooling operation. That is, the ventilation system VS1 can set the ventilation region G1, G2 suitable to the operation state in accordance with the operation state of the air conditioning device 8. As a result, it is possible to improve the comfort of the person H1 in the space R1 (keep head cool and feet warm) or make the temperature distribution in the space R1 uniform.

### (3) First Variation

The airflow-rate controller 4b of the control device 4 may control a nozzle ventilation amount which is the ventilation amount of air blown to the outside of the housing 10a through the ventilation port 10b of the nozzle 10. That is, the airflow-rate controller 4b not only controls the ventilation amount of the second conditioned air A2 (see FIG. 5) blown out through the ventilation port 3d of the ventilation device 3 but also controls the nozzle ventilation amount which is the ventilation amount of the nozzle airflow F2 (see FIG. 7) blown out through the ventilation port 10b of the nozzle 10. The airflow-rate controller 4b preferably controls such that the nozzle ventilation amount in the case of the operation state of the air conditioning device 8 being the heating operation is smaller than the nozzle ventilation amount in the case of the operation state of the air conditioning device 8 being the cooling operation.

In the following description, air in the space R1 is fed via the duct D1 to the nozzle ventilator 2, and the nozzle ventilator 2 feeds the air in the space R1 to the nozzle 10. That is, the duct D1 and the nozzle ventilator 2 circulate the air in the space R1, and the nozzle airflow F2 is circulation air obtained by circulating the air in the space R1. Moreover, the ratio of the nozzle airflow F2 included in the combination airflow F1, that is, the ratio of the circulation air included in the combination airflow F1 is defined as a mixing ratio.

The nozzle ventilation amount in the case of the operation state of the air conditioning device 8 being the heating operation is smaller than the nozzle ventilation amount in the case of the operation state of the air conditioning device 8 being the cooling operation. Thus, the mixing ratio during the heating operation is lower than the mixing ratio during the cooling operation. As a result, the temperature difference between the combination airflow F1 (the warm airflow F11 in FIG. 10) obtained by combining the nozzle airflow F2 and the draw-in airflow F3 and peripheral air (circulation air) in the space R1 is large, and thus, the comfort of the person H1 by heating can be further improved. Moreover, the nozzle ventilation amount during the heating operation is controlled to be smaller than the nozzle ventilation amount during the cooling operation, and thereby, the wind velocity of the nozzle airflow F2 decreases, and the amount of the second conditioned air A2 drawn in the nozzle airflow F2 at the upper portion of the nozzle unit 1 decreases, and thus, a satisfactory amount of the second conditioned air A2 drawn in the nozzle airflow F2 at the lower portion of the nozzle unit 1 can be secured. As a result, the second conditioned air A2 is readily drawn to the front in the vicinity of the feet of the person H1, and thereby, the warm airflow F11 can be satisfactorily sent to the vicinity of the feet of the person H1.

The nozzle ventilation amount in the case of the operation state of the air conditioning device 8 being the cooling operation is larger than the nozzle ventilation amount in the case of the operation state of the air conditioning device 8 being the heating operation. Thus, the mixing ratio during the cooling operation is higher than the mixing ratio during the heating operation. As a result, the temperature difference between the combination airflow F1 (the cool airflow F12 in FIG. 12) obtained by combining the nozzle airflow F2 and the draw-in airflow F3 and peripheral air (circulation air) in the space R1 is small, and thus, a draft sensation due to cooling can be suppressed. Moreover, the nozzle ventilation amount of the cooling operation is controlled to be larger than the nozzle ventilation amount during the heating operation, and thereby, the wind velocity of the nozzle airflow F2 increases, and thus, the amount of the second conditioned air A2 drawn in the nozzle airflow F2 at the upper portion of the nozzle unit 1 increases. As a result, the second conditioned air A2 is readily drawn to the front above the head of the person H1, and therefore, the cool airflow F12 is difficult to directly reach the person H1.

### (4) Second Variation

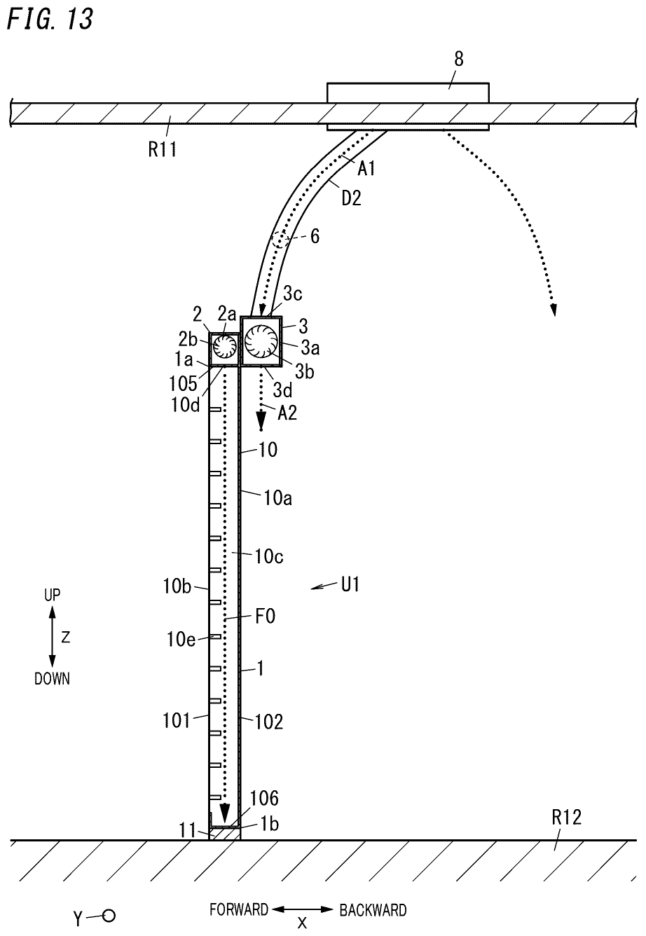

FIG. 13 shows a second variation of the embodiment. In the second variation, the ventilation system VS1 further includes a duct D2. The duct D2 is a cylindrical body that connects a duct connection port through which the air conditioning device 8 blows the first conditioned air A1 and the suction port 3c of the ventilation device 3 to each other. That is, the duct D2 connects the air conditioning device 8 to the ventilation device 3 to guide the first conditioned air Al blown out of the air conditioning device 8 to the ventilation device 3. The first conditioned air A1 blown out of the air conditioning device 8 flows in the interior of the duct D2 and is sucked into the ventilation device 3.

In the second variation, the first conditioned air Al can be efficiently fed to the ventilation device 3. Thus, the ventilation system VS1 can blow airflow (warm airflow F11, cool airflow F12) having an air conditioning effect by the air conditioning device 8 to the front of the nozzle unit 1.

### (5) Third Variation

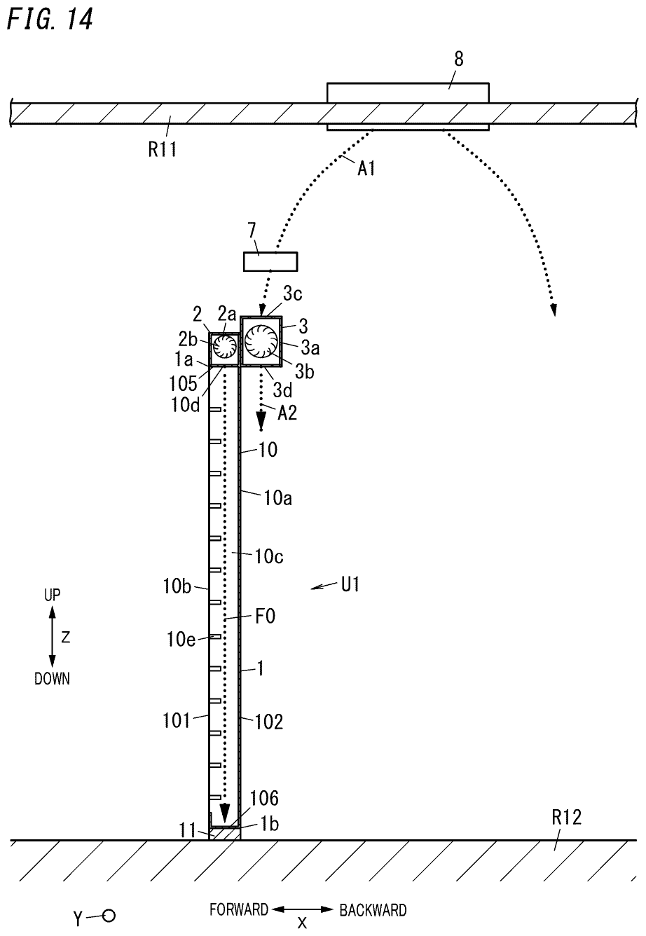

FIG. 14 shows a third variation of the embodiment. In the third variation, the ventilation system VS1 further includes an auxiliary ventilation device 7. The auxiliary ventilation device 7 is located between the air conditioning device 8 and the ventilation device 3 in the vertical direction and blows air sucked from above the auxiliary ventilation device 7 to below the auxiliary ventilation device 7. The first conditioned air Al blown out of the air conditioning device 8 is sucked via the auxiliary ventilation device 7 into the ventilation device 3.

In the third variation, the first conditioned air Al can be efficiently fed to the ventilation device 3. Thus, the ventilation system VS1 can blow airflow (warm airflow F11, cool airflow F12) having an air conditioning effect by the air conditioning device 8 to the front of the nozzle unit 1.

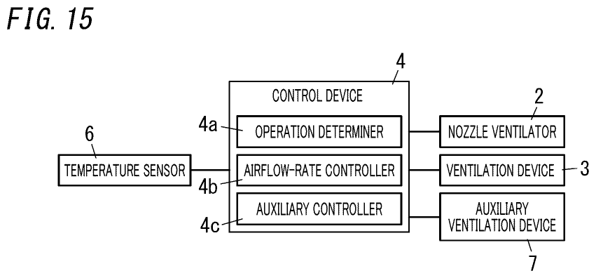

FIG. 15 shows a block diagram relating to the control by the control device 4 of the third variation. The control device 4 controls the auxiliary ventilation device 7 in addition to the ventilation device 3. Specifically, the control device 4 further includes an auxiliary control unit 4c. The auxiliary control unit 4c controls such that the auxiliary ventilation device 7 is also in the operation state at the same time as the ventilation device 3. That is, the control device 4 operates the ventilation device 3 and the auxiliary ventilation device 7 synchronously.

Moreover, the auxiliary control unit 4c may control such that the ventilation amount of the auxiliary ventilation device 7 is proportional to the ventilation amount of the ventilation device 3. Specifically, when the operation state of the air conditioning device 8 is the “heating operation”, the auxiliary control unit 4c controls such that the ventilation amount of the auxiliary ventilation device 7 is “large”, and when the operation state of the air conditioning device 8 is the “cooling operation”, the auxiliary control unit 4c controls such that the ventilation amount of the auxiliary ventilation device 7 is “small”. In this case, the auxiliary ventilation device 7, together with the ventilation device 3, can change the ventilation region of the ventilation system VS1.

### (6) Fourth Variation

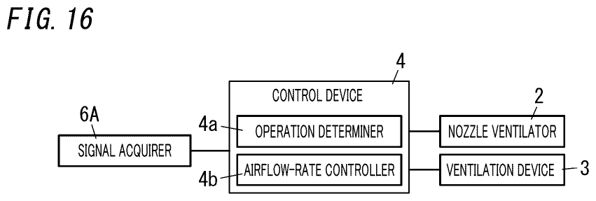

FIG. 16 shows a block diagram relating to control by the control device 4 of a fourth variation.

In the fourth variation, the ventilation system VS1 includes a signal acquirer 6A as an alternative to the temperature sensor 6. The signal acquirer 6A acquires an air conditioning operation signal for operating the air conditioning device 8. The air conditioning operation signal is a signal transmitted by wireless communication or wired communication from an operation device (an air conditioning operation device) of the air conditioning device 8 to the air conditioning device 8 and instructs the heating operation, cooling operation, and the like of the air conditioning device 8. The signal acquirer 6A monitors the air conditioning operation signal on a communication path or cause the air conditioning device 8 to transmit the air conditioning operation signal, thereby obtaining the air conditioning operation signal.

The operation determiner 4a determines the operation state of the air conditioning device 8 on the basis of the air conditioning operation signal. For example, if the air conditioning operation signal is a signal instructing the heating operation of the air conditioning device 8, the operation determiner 4a determines that the operation state of the air conditioning device 8 is the “heating operation”. If the air conditioning operation signal is a signal instructing the cooling operation of the air conditioning device 8, the operation determiner 4a determines that the operation state of the air conditioning device 8 is the “cooling operation”.

In a similar manner to the embodiment described above, when the operation state of the air conditioning device 8 is the “heating operation”, the airflow-rate controller 4b controls such that the ventilation amount of the ventilation device 3 is “large”, and when the operation state of the air conditioning device 8 is the “cooling operation”, the airflow-rate controller 4b controls such that the ventilation amount of the ventilation device 3 is “small”.

As described above, the operation determiner 4a can accurately determine the operation state of the air conditioning device 8.

### (7) Fifth Variation

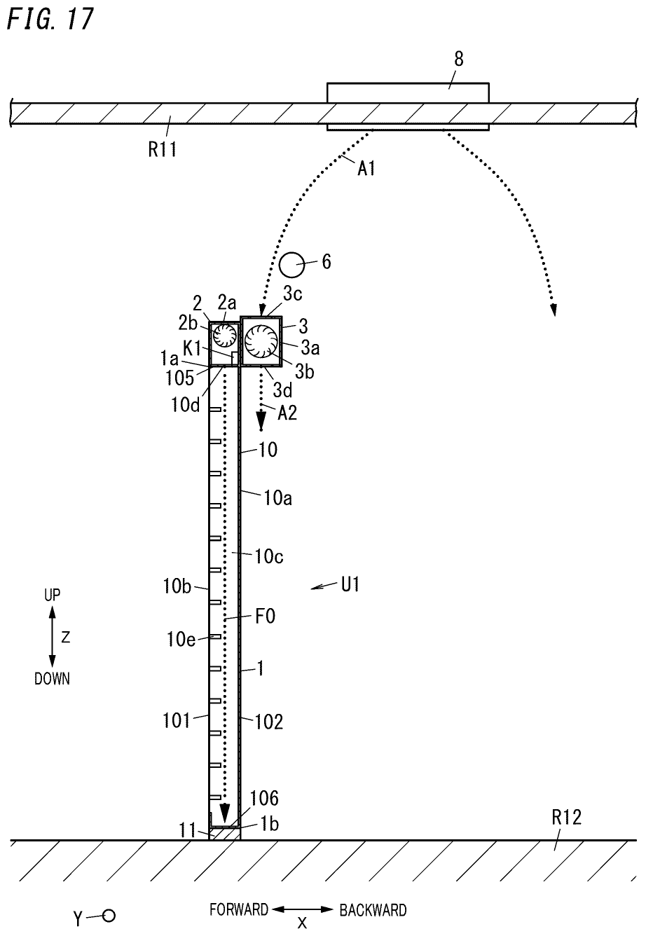

FIG. 17 shows part of the configuration of the ventilation system VS1 of a fifth variation.

The ventilation system VS1 of the fifth variation further includes an imparting device K1 which impart an effective component to air which is to be blown out through the ventilation port 10b of each nozzles 10.

In FIG. 17, the imparting device K1 is disposed in the housing 2a of the nozzle ventilator 2. The imparting device K1 imparts an effective component to air (interior airflow F0) to be sent from the nozzle ventilator 2 to the internal space 10c. As a result, the nozzle airflow F2(see FIG. 7) including the effective component blows out through the ventilation port 10b, and airflow including the effective component is generated.

For example, the imparting device K1 discharges to generate the effective component. Specifically, the imparting device K1 includes a pair of electrodes and holds water on one electrode of the pair of electrodes. The imparting device K1 applies a voltage across the pair of electrodes to cause a discharge between the pair of electrodes, thereby generating radicals as effective components and electrostatically nebulizing the water held by the electrode. Thus, the imparting device K1 generates nanometer-size charged microparticle water including radicals in a fine droplet of the electrostatically nebulized water. The radicals are basis for providing useful effects in various situations not limited to sterilization, deodorization, moisture retention, freshness retention, and virus inactivation.

Moreover, the imparting device K1 may cause a discharge between the pair of electrodes without having the electrode hold water. In this case, the imparting device K1 generates air ions as the effective component by the discharge caused between the pair of electrodes.

Moreover, the imparting device K1 may generate a fragrance component, hypochlorous acid, or the like as the effective component.

Moreover, the imparting device K1 may be disposed in the housing 3a of the ventilation device 3. In this case, the effective component is imparted to the second conditioned air A2 to be blown out of the ventilation device 3.

The airflow-rate controller 4b may control the ventilation amount of the second conditioned air A2 by the ventilation device 3 on the basis of, for example: an operation given by a user to the operation device 5 or the like; the location of the person H1 in the space R1 detected by a motion sensor; and a timing result by a timer. In this case, in the ventilation system VS1, manual control of the ventilation region, automated control based on the location of the person H1, schedule control, and the like are possible.

The nozzle ventilator 2 may be a ventilator other than the cross-flow fan, and may be, for example, a sirocco fan or a propeller fan.

Moreover, the nozzle ventilator 2 may be configured to suck in air via a duct or may be configured to suck in air around the housing 2a.

The nozzle unit 1 includes at least two nozzles 10.

Moreover, a structural component to which the ventilation unit U1 is to be attached may be a structural component, such as a ceiling or a mount, located at the upper portion in the space R1.

Moreover, each of the configurations of the embodiment and the variations described above may accordingly be combined with each other, and the effect of each configuration can likewise be obtained.

### (9) Summary

A ventilation system (VS1) of a first aspect according to the embodiment is installed in a space (R1) in which an air conditioning device (8) blows first conditioned air (A1). The ventilation system (VS1) includes a nozzle unit (1) and a ventilation device (3). The nozzle unit (1) includes at least two nozzles (10) each including a housing (10a) having a hollow elongated shape extending in a first direction, the at least two nozzles (10) being arranged side by side in alignment with a second direction intersecting the first direction. The ventilation device (3) is configured to suck in the first conditioned air (A1) and blow out second conditioned air (A2). The housing (10a) has a first surface part (101) and a second surface part (102) facing each other in a third direction intersecting the first direction and the second direction. The first surface part (101) has a ventilation port (10b) extending in the first direction. The ventilation port (10b) is configured to allow air sent into the housing (10a) to be blown to an outside of the housing (10a) through the ventilation port (10b). The ventilation device (3) is located at a side of the second surface part (102) of the housing (10a) with respect to the nozzle unit (1) and configured to blow the second conditioned air (A2) from a first end (1a) toward a second end (1b) in the first direction of the nozzle unit (1).

The ventilation system (VS1) enables a ventilation region (G1, G2) to be changed with a simplified structure of each nozzle (10).

A ventilation system (VS1) of a second aspect according to the embodiment referring back to the first aspect preferably further includes an airflow-rate controller (4b) configured to control a ventilation amount of the second conditioned air (A2) by the ventilation device (3).

The ventilation system (VS1) enables a ventilation region (G1, G2) to be changed with a simplified structure of each nozzle (10).

A ventilation system (VS1) of a third aspect according to the embodiment referring back to the second aspect preferably further includes an operation determiner (4a) configured to determine an operation state of the air conditioning device (8). The airflow-rate controller (4b) is configured to control, based on a determination result of the operation state, the ventilation amount of the second conditioned air (A2) by the ventilation device (3).

The ventilation system (VS1) can send airflow (F11, F12) to the ventilation region (G1, G2) according to the operation state of the air conditioning device (8).

In a ventilation system (VS1) of a fourth aspect according to the embodiment referring to the third aspect, the operation determiner (4a) is preferably configured to determine whether the operation state is heating operation or cooling operation.

The ventilation system (VS1) can send streams of airflow (F11, F12) respectively to the ventilation regions (G1, G2) according to the heating operation and the cooling operation of the air conditioning device (8).

In a ventilation system (VS1) of a fifth aspect according to the embodiment referring to the fourth aspect, the airflow-rate controller (4b) is preferably configured to control such that the ventilation amount in a case of the operation state being the heating operation is larger than the ventilation amount in a case of the operation state being the cooling operation.

The ventilation system (VS1) can send streams of airflow (F11, F12) respectively to the ventilation regions (G1, G2) according to the heating operation and the cooling operation of the air conditioning device (8).

In a ventilation system (VS1) of an eighth aspect according to the embodiment referring to the fourth aspect, the airflow-rate controller (4b) is preferably configured to control a nozzle ventilation amount which is a ventilation amount of air blown to an outside of the housing (10a) through the ventilation port (10b). The airflow-rate controller (4b) is configured to control such that the nozzle ventilation amount in the case of the operation state being the heating operation is smaller than the nozzle ventilation amount in the case of the operation state being the cooling operation.

The ventilation system (VS1) can improve thermal comfort felt by a person in the space (R1).

A ventilation system (VS1) of each of thirteenth, eleventh, sixth, and ninth aspects according to the embodiment referring to any one of the third to sixth aspects preferably further includes a temperature sensor (6) configured to detect a temperature of the first conditioned air (A1). The operation determiner (4a) is configured to determine the operation state on a basis of the temperature of the first conditioned air (A1).

The ventilation system (VS1) can accurately determine the operation state of the air conditioning device (8).

A ventilation system (VS1) of each of fourteenth, twelfth, seventh, and tenth aspects according to the embodiment referring to any one of the third to sixth aspects preferably further includes a signal acquirer (6A) configured to acquire an air conditioning operation signal for operating the air conditioning device (8). The operation determiner (4a) is configured to determine the operation state on a basis of the air conditioning operation signal.

The ventilation system (VS1) can accurately determine the operation state of the air conditioning device (8).

A ventilation system (VS1) of a fifteenth aspect according to the embodiment referring to any one of the first to fourteenth aspects preferably further includes a duct (D2) configured to guide the first conditioned air (A1) blown out of the air conditioning device (8) to the ventilation device (3).

The ventilation system (VS1) can blow airflow (F11, F12) having an air conditioning effect by the air conditioning device (8).

A ventilation system (VS1) of a sixteenth aspect according to the embodiment referring to any one of the first to fourteenth aspect preferably further includes an auxiliary ventilation device (7) configured to suck in the first conditioned air (A1) blown out of the air conditioning device (8) and blow the first conditioned air (A1) toward the ventilation device (3).

The ventilation system (VS1) can blow airflow (F11, F12) having an air conditioning effect by the air conditioning device (8).

A ventilation system (VS1) of a seventeenth aspect according to the embodiment referring to any one of the first to sixteenth aspects preferably further includes an imparting device (K1) configured to impart an effective component to air.

The ventilation system (VS1) can blow air including an effective component such as a radical, fragrance, hypochlorous acid, or the like and can thus improve the environment of the space (R1).

In a ventilation system (VS1) of an eighteenth aspect according to the embodiment referring to any one of the first to seventeenth aspects, the first direction is preferably a vertical direction.

In the ventilation system (VS1), the nozzle unit (1) can be used also as a partition.

In a ventilation system (VS1) of a nineteenth aspect according to the embodiment referring to the eighteenth aspect, the first end (1a) is preferably located above the second end (1b).

The ventilation system (VS1) can improve the comfort of the person in the space (R1) or make the temperature distribution in the space (R1) uniform.

### REFERENCE SIGNS LIST

VS1 Ventilation System
1 Nozzle Unit
1a Upper End (First End)
1b Lower End (Second End)
10 Nozzle
101 First Surface
102 Second Surface
10a Housing
10b Ventilation Port
3 Ventilation Device
4a Operation Determiner
4b Airflow-Rate Controller
6 Temperature Sensor
6A Signal Acquirer
7 Auxiliary Ventilation Device
8 Air Conditioning Device
A1 First Conditioned Air
A2 Second Conditioned Air
D2 Duct
K1 Imparting Device

1. A ventilation system installed in a space in which an air conditioning device blows first conditioned air, the ventilation system comprising:
a nozzle unit including at least two nozzles each including a housing having a hollow elongated shape extending in a first direction, the at least two nozzles being arranged side by side in alignment with a second direction intersecting the first direction;
a ventilation device configured to suck in the first conditioned air and blow out second conditioned air,
the housing having a first surface part and a second surface part facing each other in a third direction intersecting the first direction and the second direction,
the first surface part having a ventilation port extending in the first direction,
the ventilation port being configured to allow air sent into the housing to be blown to an outside of the housing through the ventilation port,
the ventilation device being located at a side of the second surface part of the housing with respect to the nozzle unit and being configured to blow the second conditioned air from a first end toward a second end in the first direction of the nozzle unit.

2. The ventilation system of claim 1, further comprising an airflow-rate controller configured to control a ventilation amount of the second conditioned air by the ventilation device.

3. The ventilation system of claim 2, further comprising an operation determiner configured to determine an operation state of the air conditioning device, wherein
the airflow-rate controller is configured to control, based on a determination result of the operation state, the ventilation amount of the second conditioned air.

4. The ventilation system of claim 3, wherein
the operation determiner is configured to determine whether the operation state is heating operation or cooling operation.

5. The ventilation system of claim 4, wherein
the airflow-rate controller is configured to control such that the ventilation amount in a case of the operation state being the heating operation is larger than the ventilation amount in a case of the operation state being the cooling operation.

6. The ventilation system of claim 5, further comprising a temperature sensor configured to detect a temperature of the first conditioned air, wherein the operation determiner is configured to determine the operation state on a basis of the temperature of the first conditioned air.

7. The ventilation system of claim 5, further comprising a signal acquirer configured to acquire an air conditioning operation signal for operating the air conditioning device, wherein
the operation determiner is configured to determine the operation state on a basis of the air conditioning operation signal.

8. The ventilation system of claim 4, wherein
the airflow-rate controller is configured to
control a nozzle ventilation amount which is a ventilation amount of air blown to an outside of the housing through the ventilation port and
control such that the nozzle ventilation amount in the case of the operation state being the heating operation is smaller than the nozzle ventilation amount in the case of the operation state being the cooling operation.

9. The ventilation system of claim 8, further comprising a temperature sensor configured to detect a temperature of the first conditioned air, wherein
the operation determiner is configured to determine the operation state on a basis of the temperature of the first conditioned air.

10. The ventilation system of claim 8, further comprising a signal acquirer configured to acquire an air conditioning operation signal for operating the air conditioning device, wherein
the operation determiner is configured to determine the operation state on a basis of the air conditioning operation signal.

11. The ventilation system of claim 4, further comprising a temperature sensor configured to detect a temperature of the first conditioned air, wherein the operation determiner is configured to determine the operation state on a basis of the temperature of the first conditioned air.

12. The ventilation system of claim 4, further comprising a signal acquirer configured to acquire an air conditioning operation signal for operating the air conditioning device, wherein
the operation determiner is configured to determine the operation state on a basis of the air conditioning operation signal.

13. The ventilation system of claim 3, further comprising a temperature sensor configured to detect a temperature of the first conditioned air, wherein
the operation determiner is configured to determine the operation state on a basis of the temperature of the first conditioned air.

14. The ventilation system of claim 3, further comprising a signal acquirer configured to acquire an air conditioning operation signal for operating the air conditioning device, wherein
the operation determiner is configured to determine the operation state on a basis of the air conditioning operation signal.

15. The ventilation system of claim 1, further comprising a duct configured to guide the first conditioned air blown out of the air conditioning device to the ventilation device.

16. The ventilation system of claim 1, further comprising an auxiliary ventilation device configured to suck in the first conditioned air blown out of the air conditioning device and blow out the first conditioned air toward the ventilation device.

17. The ventilation system of claim 1, further comprising an imparting device configured to impart an effective component to the air.

18. The ventilation system of claim 1, wherein
the first direction is a vertical direction.

19. The ventilation system of claim 18, wherein
the first end is located above the second end.
